(19) United States
(12) Patent Application Publication
(10) Pub. No.: US 2026/0306431 A1
(43) Pub. Date: Oct. 8, 2026

(54) SULFURIZED IMIDAZOLES IN OPHTHALMIC FORMULATIONS

(71) Applicant: GENEIII BIOTECH COMPANY LTD., Nanjing (CN)

(72) Inventor: Cai Rong Wang, Hacienda Heights, CA (US)

(21) Appl. No.: 19/172,995

(22) Filed: Apr. 8, 2025

(51) Int. Cl. A61K 31/198; A61K 9/00; A61K 31/047; A61K 31/122; A61K 38/06; A61K 45/06; A61K 47/18; A61P 27/02

(52) U.S. Cl. CPC A61K 31/198; A61K 9/0048; A61K 31/047; A61K 31/122; A61K 38/063; A61K 45/06; A61K 47/183; A61P 27/02

(57) ABSTRACT

In the embodiments, ophthalmic formulations comprise 0.001-2% (w/v) of L-Ergothioneine or derivatives of L-Ergothioneine, optionally combined with compatible toxicity agents. L-ergothioneine or derivatives of L-Ergothioneine in the ophthalmic formulation is: (1) an active pharmaceutical ingredient (API) for treating oxidative stress-related ocular disorders; and (2) an inactive excipient for use as a stabilizing excipient to enhance bioavailability and prolong shelf-life of co-administered APIs. L-ergothioneine or derivatives of L-ergothioneine in the ophthalmic solution, when applied on an ocular surface, exhibit: improved corneal permeability; reduced degradation of thermolabile components; and synergistic therapeutic effects when used with other neuroprotective agents, as the co-formulated APIs, in the ophthalmic formulations.

### BACKGROUND OF THE INVENTION

### 1. Field of the Invention

The compositions and methods herein are directed to pharmaceutical formulations for treating ocular diseases or alleviating symptoms associated with ocular diseases.

### 2. Description of Prior Art and Related Information

Over the past several decades, more than a dozen organic compounds have been developed and used as active ingredients of eye drops. Many active ingredients and preservatives have upper concentration level limits based on the considerations of toxicities for certain formulations. The search for active pharmaceutical ingredients and inactive excipients of eye drops with higher potency and lower toxicity is a focus of attention. Currently, most eye drops on the market are formulated with specific active ingredients for specific effects and can be divided into four categories: treatment of redness in eye, lubricating, tears in the eye, and allergies in the eyes.

What is need is the compositions and methods herein is an ophthalmic formulation containing L-Ergothioneine or derivatives of L-Ergothioneine (e.g., the application of L-Ergothioneine in eye drops), that are loaded at a concentration lower than the upper concentration level limits, thereby avoiding potentially toxic levels, while exhibiting higher potency in: (1) increasing lubrication; and (2) reducing redness, tears, and allergies.

### SUMMARY OF THE INVENTION

In a variant, an ophthalmic formulation comprises: a pharmaceutically acceptable aqueous delivery vehicle; and L-Ergothioneine (EGT) in a concentration of 0.005% to 2.000% (w/v) in the pharmaceutically acceptable aqueous delivery vehicle, wherein the EGT is: an active ingredient for treating an oxidative stress-related ocular disorder or disease; or an inactive excipient to inhibit degradation of a co-formulated active ingredient.

In a variant, the co-formulated active ingredient is selected from the group consisting of: nucleotides, acetylcholine, tetraethylammonium chloride, spermine, L-carnitine, sulpiride, homostachydrine, cytarabine, gemcitabine, gabapentin, oxaliplatin, metformin, cyclosporine, and lifitegrast.

In a variant, EGT scavenges hydroxyl and peroxynitrite via a non-enzymatic redox cycle, such that: the hydroxyl is reduced to water and EGT is oxidized to a sulfate; and the peroxynitrite is reduced to nitrate and EGT is oxidized to a sulfate.

In a variant, EGT coordinates to a metal selected from iron, copper, and other transition metals in an ocular surface, thereby inhibiting catalysis of oxidative degradation of labile drugs by: forming a complex between EGT and the metal, wherein the metal is in a reduced state; and reducing an oxidation potential of the metal due to the formation of the complex, thereby suppressing formation of metal-oxygen adducts for deriving reactive oxygen species; wherein the complex comprises: (i) two units of EGT binding to 1 unit of the metal or (ii) 1 unit of EGT and 1 unit of an initial ligand core binding to 1 unit of the metal.

In a variant, EGT as the inactive excipient in the formulation is reducing reactive oxygen species (ROS) in corneal epithelial cells.

In a variant, EGT is 0.5% to 1.0% (w/v) of the formulation, when functioning as an active ingredient; EGT is 0.005% to 0.5% (w/v) of the formulation, when functioning as an inactive excipient.

In a variant, the oxidative stress-related ocular disorder or disease comprises: allergies, irritated eyes, red eye, age-related macular degeneration; cataracts; glaucoma; diabetic retinopathy; dry eye syndrome; and retinitis pigmentosa.

In a variant, the co-formulated active ingredient is transported in the ocular tissue by OCTN1, thereby increasing bioavailability of the co-formulated active ingredient in the ocular tissue, wherein OCTN1 is inductively expressed by presence of EGT introduced into the ocular tissue.

In a variant, the formulation further comprises one or more of: glutathione, Ubiquinone, lutein; and ethylenediaminetetraacetic acid.

In a variant, EGT has a chemical formula of C₉H₁₅N₃O₂S, where the carbon-sulfur bond is in thione form (C═S) over thiolate form (C—S⁻), in the pharmaceutically acceptable aqueous delivery vehicle.

In a variant, the formulation comprises: 0.5% (w/v) of EGT as the active ingredient; 0.05% (w/v); 0.01% (w/v), and 0% (w/v) of sodium hyaluronate; trace amounts of taurine; sodium chloride for adjusting the osmotic pressure of eye drops; and the pharmaceutically acceptable aqueous delivery vehicle.

In a variant, an ophthalmic formulation comprises: a pharmaceutically acceptable
aqueous delivery vehicle; and a derivative of L-Ergothioneine (EGT) in a concentration of 0.005% to 3.000% (w/v) in the pharmaceutically acceptable aqueous delivery vehicle, wherein the derivative of EGT is: an active ingredient for treating an oxidative stress-related ocular disorder or disease; or an inactive excipient to inhibit degradation of a co-formulated active ingredient, wherein the derivative of EGT is:

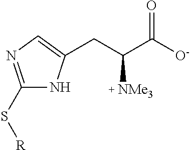

where R═CH₃, CH₂CH₃, CH₂CH₂CH₃, CH₃CHCH₃, CH₂—X, and X═1-indole, 4-Indole, 7-indole, 1-imidazole, 2-imidazole, 3-imidazole, 4-imidazole, 5-imidazole, benzene, 2-oxazole, 3-oxazole, 4-oxazole, 2-thiazole, 3-thiazole, 4-thiazole, 2-pyridine, 6-pyridine, 1-pyrrole, 2-pyrrole, and 3-pyrrole.

In a variant, wherein the co-formulated active ingredient is selected from the group consisting of: nucleotides, acetylcholine, tetraethylammonium chloride, spermine, L-carnitine, sulpiride, homostachydrine, cytarabine, gemcitabine, gabapentin, oxaliplatin, metformin, cyclosporine, and lifitegrast.

In a variant, the derivative of EGT scavenges hydroxyl, such that the hydroxyl is reduced to water and the derivative of EGT is oxidized to a sulfoxide.

In a variant, the derivative of EGT as the inactive excipient in the formulation is decreasing reactive oxygen species (ROS) in corneal epithelial cells.

In a variant, the derivative of EGT is 1.0% to 3.0% (w/v) of the formulation, when functioning as an active ingredient; and the derivative of EGT is 0.005% to 1.0% (w/v), when functioning as an inactive excipient.

In a variant, the oxidative stress-related ocular disorder or disease comprises: allergies, irritated eyes, red eye, age-related macular degeneration; cataracts; glaucoma; diabetic retinopathy; dry eye syndrome; and retinitis pigmentosa.

In a variant, a method of treating an oxidative stress-related ocular condition, disorder, or disease in a subject, wherein the method comprises: administering a formulation to said subject, wherein the formulation comprises L-Ergothioneine (EGT) or derivative of EGT in a concentration of 0.005% to 2.000% (w/v) in a pharmaceutically acceptable aqueous delivery vehicle, wherein: EGT or the derivative of EGT is: an active ingredient for treating an oxidative stress-related ocular disorder or disease; or an inactive excipient to inhibit degradation of a co-formulated active ingredient.to the ocular surface of the subject.

In a variant, EGT or the derivative of EGT scavenges reactive oxygen species via a non-enzymatic redox cycle.

In a variant, wherein EGT or the derivative of EGT coordinates to a metal selected from iron, copper, and other transition metals in an ocular surface, thereby inhibiting catalysis of oxidative degradation of labile drugs and decreasing reactive oxygen species (ROS) in corneal epithelial cells.

### BRIEF DESCRIPTION OF THE DRAWINGS

FIG. 1 is a depiction of canonical structures of L-Ergothioneine (EGT).

FIG. 2 is a depiction of EGT and derivatives of EGT and the scavenging of reactive oxygen species.

FIG. 3 is a depiction of EGT binding to free metal ions.

FIG. 4 is a depiction of EGT binding to iron from lipoxygenase.

FIG. 5 is a depiction of EGT binding to iron from Cytochrome P450.

FIG. 6 is a depiction of a questionnaire plot.

FIG. 7 is a depiction of a OSDI Rating Scale/Score plot.

FIG. 8 is a depiction of a visual fatigue scale/score plot.

FIG. 9 is a depiction of the first tear film beak-up time plot for the left eye.

FIG. 10 is a depiction of the first tear film beak-up time plot for the right eye.

### DETAILED DESCRIPTION OF THE PREFERRED EMBODIMENTS AND BEST MODE OF INVENTION

In the compositions and methods herein, the ophthalmic formulation herein can be introduced into a physiological environment, as eye drops added to ocular tissue of a human subject. The ophthalmic formulation herein comprises: (1) a pharmaceutically acceptable aqueous delivery vehicle (e.g., water as a solvent for dissolving tonic agents, buffers, preservatives, viscosity enhancers, stabilizers, pH adjusters, penetration enhancers, local anesthetics, restorative agents, surfactants, and thickeners); (2) L-Ergothioneine (EGT) or derivatives of EGT dissolved in the water phase of the pharmaceutically acceptable aqueous delivery vehicle; (3) one or more co-formulated active ingredients (nucleotides (e.g., uridine), acetylcholine, tetraethylammonium chloride, spermine, L-carnitine, sulpiride, homostachydrine, cytarabine, gemcitabine, gabapentin, oxaliplatin, metformin, cyclosporine, and Lifitegrast); and (4) other ingredients selected from one or more of: (a) sodium chloride or other sodium salts for controlling osmotic pressure; (b) stabilizers (ethylenediaminetetraacetic acid); (c) hyaluronates; (d) taurine (2-aminoethanesulfonic acid); and (e) co-antioxidants (glutathione, ubiquinone, and lutein).

In the compositions and methods herein, EGT or derivatives of EGT are introduced to ocular tissue via an ophthalmic formulation, such as eye drops. EGT or derivatives of EGT in the ophthalmic formulation can behave as: (1) an active pharmaceutical ingredient (API) for treating or alleviating symptoms associated with oxidative stress-related ocular disorders (e.g., allergies, irritated eyes, red eye, age-related macular degeneration, cataracts, glaucoma, diabetic retinopathy, dry eye syndrome; and retinitis pigmentosa, and UV-induced corneal damage); and (2) an inactive excipient for use, as a stabilizing excipient, to: (a) enhance the bioavailability of co-administered active pharmaceutical ingredients; (b) prolong the shelf-life of co-administered active pharmaceutical ingredients; (c) increase the stability of the ophthalmic formulation herein; and (d) increase compatibility of EGT or EGT derivatives with other ocular therapeutics. The combination of API and excipient behavior of EGT or derivatives of EGT can: improve the potency of eye drops and reduce the toxicity of eye drops, in comparison to eye drops absent of EGT or derivatives of EGT. This can be attributed to: reduction of irritating excipients; the low-toxicity, antioxidant, and anti-inflammatory properties of EGT or derivative of EGT; and the clinical safety, which is described below.

In the compositions and methods herein, the ophthalmic formulations herein, which contain EGT or derivatives of EGT in the pharmaceutically acceptable aqueous delivery vehicle have a viscosity, surface activity, and surface tension adjustments that aid in the targeted delivery of EGT or derivatives of EGT to reduce oxidative stress in corneal epithelial cells. The amount of EGT or derivatives of EGT in the ophthalmic formulation herein can determine whether EGT or a derivative of EGT is an API or excipient. For example, the concentration of EGT in the ophthalmic formulation herein is: (1) 0.5% to 1.0% (w/v), when functioning as an active pharmaceutical ingredient; or (2) 0.005% to 0.5% (w/v) when functioning as an inactive excipient. For example, the ophthalmic formulations herein comprise: 98.0-99.5% (w/v) of water; 0.1-0.5% (w/v) of triethylamine; 0.05-2.0% (w/v) of L-carnitine; 0.01-0.1% (w/v) of Spermine, and 0.1-1.0% (w/v) of acetylcholine, in combination with: (1) 0.5% to 1.0% (w/v); or (2) 0.005% to 0.5% (w/v) of EGT. EGT is dissolved in the pharmaceutically acceptable aqueous delivery vehicle, which is predominantly water and contributes at least 85 to 90% (w/v) water to the formulation. The water content of the pharmaceutically acceptable aqueous delivery vehicle dissolves one or more of a tonic agent, buffer, preservative, viscosity enhancer, stabilizer, pH adjuster, penetration enhancer, local anesthetic, restorative agent, surfactant, and thickeners, to (1) make the pharmaceutically acceptable aqueous delivery vehicle, to which (2) EGT is added. To achieve the same level of alleviation of ocular symptoms, lower amounts of EGT or derivatives of EGT are used in the ophthalmic formulations herein, in comparison to other API eye treatments, especially with the consideration of the same toxicity level.

In the compositions and methods herein, EGT or derivatives of EGT can perform the functions of: (1) scavenging reactive oxygen species (ROS), such as free radicals (e.g., hydroxyl radical) and free radical surrogates (e.g., peroxynitrite (ONOO⁻) or peroxynitrous acid (ONOOH)), more effectively than butylated hydroxytoluene (BHT) and 1,2,3-benzotriazole (BHA), with minimal cytotoxicity; (2) exhibiting strong antioxidant activity to consume ROS that can oxidatively degrade co-formulated active ingredients; (3) inhibiting inflammation, thereby making the ophthalmic formulation herein the particularly suitable for coping with corneal oxidative stress caused by light loss and dry eye syndrome; (4) exhibiting dual-phase stabilization to inhibit degradation of labile active ingredients (e.g., pharmaceutical ingredients prone to chemical degradation (e.g., oxidation, hydrolysis, and photolysis) or changes in physical properties (e.g., aggregation and crystallization)) in aqueous solutions, without additional preservatives, such that two immiscible phases maintain interface stability between a non-polar phase (oil) and polar phase (water) with emulsifiers and stabilizer, as exhibited in micelles, emulsions, and/or interactions between: (a) an inner mucus layer and middle aqueous layer and (b) an outer lipid layer and middle aqueous layer, in tear films; and (5) enhancing bioavailability of the co-formulated activate ingredients, via the affinity of EGT for OCTN1 transporters in corneal epithelial cells (i.e., EGT induced expression of OCTN1 transporters), thereby facilitating targeted delivery of co-formulated active ingredients (i.e., transporting the co-formulated active ingredient in ocular tissue).

In the composition and methods herein, the preferred embodiment of the ophthalmic formulations herein comprises EGT. More particularly, 0.001-2.000% (w/v) of L-Ergothioneine (EGT) can be optionally combined with compatible toxicity agents. Compatible cytotoxicity agents are a combination of ingredients that coexist in a preparation, such as tablet, eye drop, or other formulation, associated with a certain degree of toxicity. The combination of ingredients require specific formulation design to ensure chemical and physical compatibility, while managing toxicity. For example, 0.005-0.01% (w/v) of BAK and 0.01-0.05% (w/v) of EDTA can reduce metal ion oxidizing and increase antibacterial effects.

In the compositions and methods herein, other embodiments of the ophthalmic formulations herein comprise derivatives of EGT. More particularly, 0.002-3.000% (w/v) of derivatives of EGT can be optionally combined with compatible toxicity agents. A higher amount of derivatives of EGT, in comparison to EGT, may be necessary to achieve the same therapeutic effect of EGT. This may be attributed to an active group of the EGT derivatives being obscured and not compensated by other mechanisms, such as targeting; and/or efficacy enhancement by optimizing pharmacokinetics.

In the compositions and methods herein, EGT or derivatives of EGT in the ophthalmic solution, when applied on an ocular surface, lead to: improved corneal permeability; reduced degradation of thermolabile components; and synergistic therapeutic effects when used with other neuroprotective agents in the ophthalmic formulations. The thermolabile components (proteins and peptide drugs, certain antibiotics, probiotics, nucleic acid drugs) are prone to degradation, inactivation, or physical property changes, at high temperature environments (e.g., addition of heat). Neuroprotective agents protect neural cells from damage induced by glutamine or oxidative stress.

In the compositions and methods herein, the ophthalmic formulations herein can alternatively comprise EGT, in combination with other antioxidants (e.g., glutathione or lutein) for synergistic effects, in treating oxidative-stress related conditions in ocular tissue.

In the compositions and methods herein, the ophthalmic formulations herein can alternatively comprise EGT, in combination with stabilizers (e.g., ethylenediaminetetraacetic acid (EDTA)), as a preventative replacement in multi-dose eye drops.

In the compositions and methods herein, the ophthalmic formulations herein can alternatively be reformulated as nasal sprays or intraocular injections.

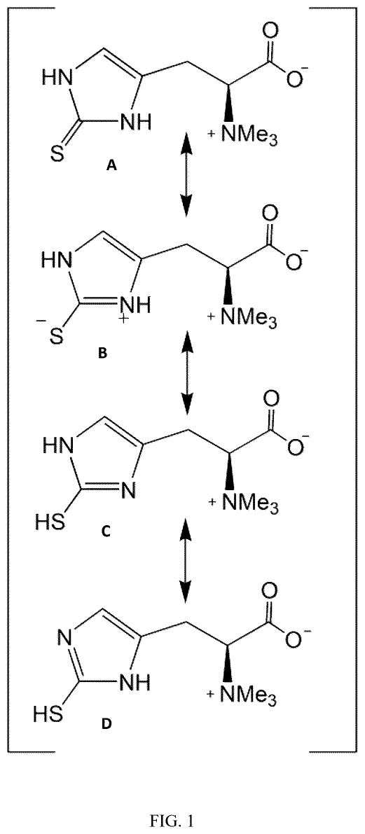

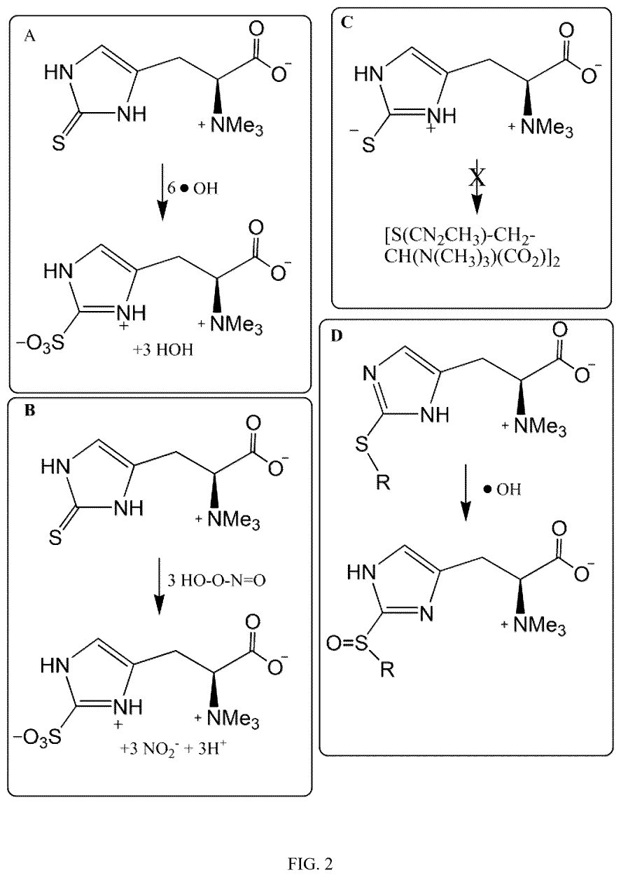

In the compositions and methods herein, the ophthalmic formulations herein comprising EGT are a preferred embodiment, wherein EGT has a thiourea group, which indicates that a thione (C═S, which is A in FIG. 1) canonical structure is favored over thiol (C—SH, which is C and D in FIG. 1) or thiolate (C—S⁻, which is B in FIG. 1) canonical structures, thereby allowing for the EGT to be an active pharmaceutical ingredient and an inactive excipient. Without being bound to a theory, thiourea of EGT, which has C═S, can scavenge of hydroxyl radicals, peroxynitrite, and peroxynitrous acid, via a non-enzymatic redox cycle. Enzymes, such as lipoxygenase and cytochrome P450, can form metal-oxygen adducts, to derive reactive oxygen species (e.g., hydroxyl radicals, peroxynitrite, and peroxynitrous acid) that react with substrates, which lead to oxidative degradations of said substrates that is associated with oxidative stress via a redox cycle. The scavenging of, for example, hydroxyl radicals and peroxynitrous acid, via non-enzymatic redox cycle, as made operable by EGT, consumes hydroxyl radicals and peroxynitrous acid to: (1) convert the thione (C═S) to sulfate (C—SO₃⁻) and obtain water (see A in FIG. 2); and (2) convert the thione (C═S) to sulfate (C—SO₃−) and obtain nitrate (NO₂⁻), and protons (see B in FIG. 2), respectively. If the canonical structure of EGT is not thione, but instead a thiolate, the observed consumption of ROS would not occur. Thiolate, such as C in FIG. 2, is an anion that has a high oxidation potential to form a thiyl radical that favors formation of disulfide. Said thiyl radical is not as readily attainable in thione. Disulfide is not observed, when the ophthalmic formulation herein is added to an ocular tissue, which suggests that the thione of a thiourea (C═S) is scavenging the ROS.

In the compositions and methods herein, the ophthalmic formulation comprises a derivative of EGT. Derivatives of EGT have the formula of RS(C(N)(NH)(CHC))—CH₂—CH(N(CH₃)₃)(CO₂), where R═CH₃, CH₂CH₃, CH₂CH₂CH₃, CH₃CHCH₃, CH₂—X, and X=1-indole, 4-Indole, 7-indole, 1-imidazole, 2-imidazole, 3-imidazole, 4-imidazole, 5-imidazole, benzene, 2-oxazole, 3-oxazole, 4-oxazole, 2-thiazole, 3-thiazole, 4-thiazole, 2-pyridine, 6-pyridine, 1-pyrrole, 2-pyrrole, and 3-pyrrole. EGT has the formula of S(C(NH)(NH)(CHC))—CH₂—CH(N(CH₃)₃)(CO₂). Derivatives of EGT are thioethers, which have a more similar valence to thione than thiolate, that scavenge ROS to yield sulfoxide (RS(O)—(C(N)(NH)(CHC))—CH₂—CH(N(CH₃)₃)(CO₂), see D of FIG. 2). Without being bound to a theory, the valence of the sulfur attached to the imidazole ring can impact the oxidation of the sulfur, which leads to the scavenging of ROS.

In the compositions and methods herein, the thione (C═S) of the EGT of the ophthalmic formulation can suppress the formation of ROS that lead to the oxidative degradation of labile drugs, such as co-formulated active ingredients, in the ophthalmic formulation. Without being bound to a theory, the sulfur of the thione is a donating atom that can form chelation complexes with a metal center. This chelation complex lowers the oxidation potential of the chelated metal center, thereby suppressing redox processes that lead to the formation of metal oxygen adducts for deriving ROS.

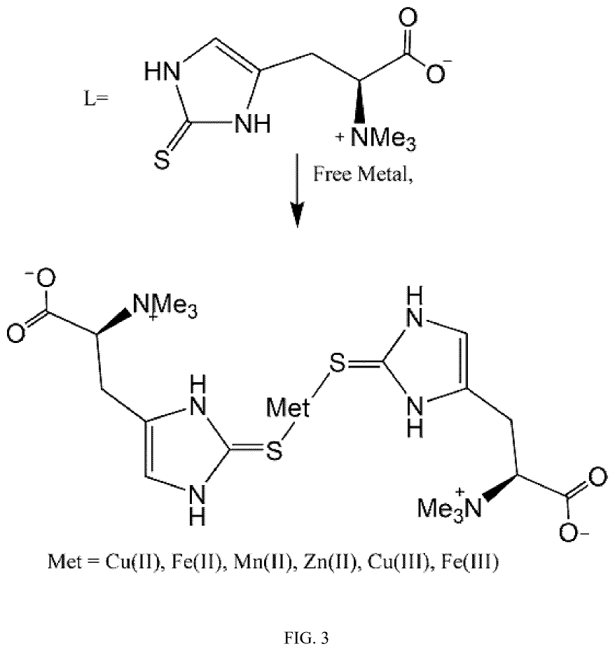

In the compositions and methods herein, two units of EGT can bind to free ions, such as Cu(II), Fe(II), Mn(II), Zn(II), Cu(III), and Fe(III), wherein the sulfur of the thione (C═S) is binding to said free ions, as depicted in FIG. 3. The chelation complex is FIG. 3 does not have sufficient oxidation potential to form metal-oxygen adducts for deriving ROS.

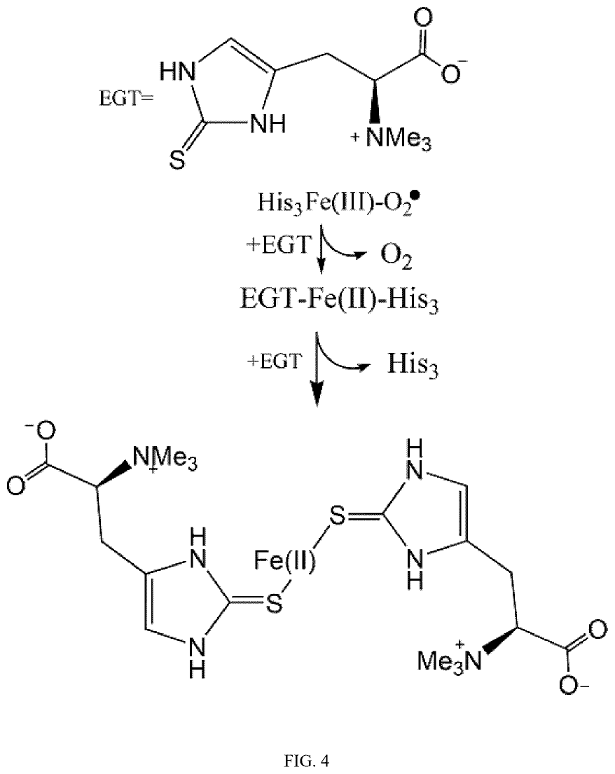

In the compositions and methods herein, two units of EGT can bind to Fe(II) from lipoxygenase-superoxide (His₃-Fe(III)-O₂•), thereby suppressing the persistence of a metal-oxygen adduct for deriving or behaving as a ROS. More particularly, the sulfur of the thione (C═S) from the first unit of EGT induces release of dioxygen, with concurrent reduction from Fe(III) to Fe(II), when the first unit of EGT binds to the iron center, to yield His₃-Fe(II)-EGT. The second unit of EGT binds to the Fe(II) site, thereby de-ligating the histidine core from the iron in lipoxygenase, to yield EGT-Fe(II)-EGT, as depicted in FIG. 4. The histidine core of lipoxygenase is a strong nitrogen-atom donating ligand for binding to Fe(II) and imparting increased oxidation potential that readily forms metal-oxygen adducts that lead to ROS. The chelation complex containing two units of EGT bound to Fe(II), as depicted in FIG. 4, does not form metal oxygen adducts. This suggests that: (1) EGT can behave as a ligand that binds to Fe(II) over the histidine core of lipoxygenase; and (2) and the two units of EGT lowers the oxidation potential of Fe(II), to suppress redox processes that form metal oxygen adducts for deriving ROS.

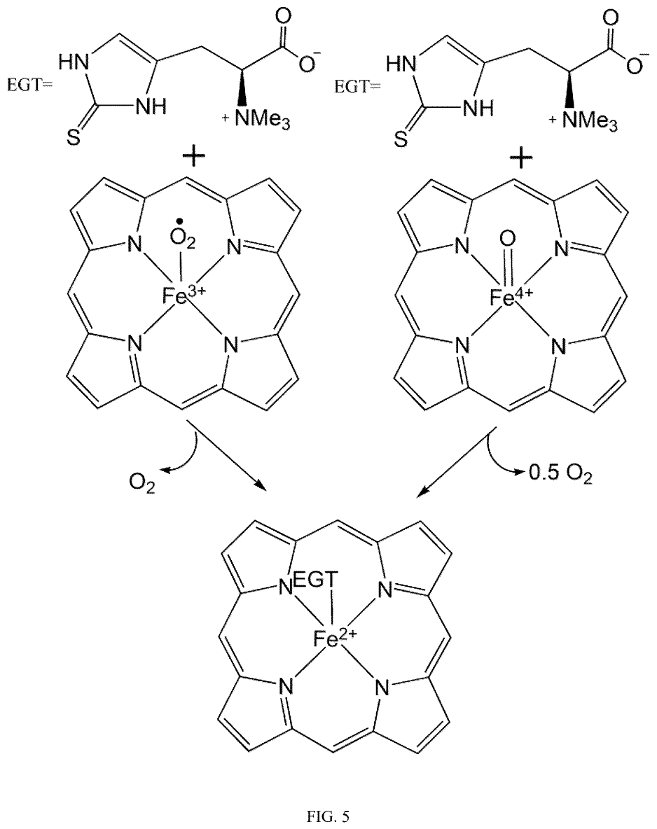

In the composition and methods herein, a unit of EGT can bind to Fe(II) from Cytochrome P450, thereby suppressing the persistence of a metal-oxygen adduct that can behave as or derive ROS. Cytochrome P450 has a porphyrin core around Fe, as depicted in FIG. 5, wherein Fe can bind to a single unit of EGT. When a metal oxygen adduct is formed in cytochrome P450, the Fe(III)-superoxide or Fe(IV)=O can form, which are ROS or derive ROS that oxidatively degrade labile drugs or lead to oxidative stress in the ocular tissue. While the porphyrin core is not readily de-ligated, the sulfur atom of the thione of EGT can bind to an oxidized iron site (Fe(III) or Fe(IV)) to release dioxygen, and concurrently reduce the iron site to Fe(II), thereby forming a chelation complex of EGT-Fe(II). The chelation complex of EGT-Fe(II) lowers the oxidation potential of Fe(II), to suppress redox processes that form metal oxygen adducts for deriving ROS.

In the compositions and methods herein, the ophthalmic formulation comprises EGT and co-formulated active ingredients, wherein EGT induces expression of transporter OCTN1. Transporter OCTN1 can transport co-formulated active ingredients to ocular tissue, when the ophthalmic formulation comprising EGT and co-formulated active ingredients is introduced into ocular tissue. This transport of co-formulated active ingredients to ocular tissue is associated with the increase of the bioavailability of the co-formulated active ingredients in the ocular tissue. Additionally, EGT can induce upregulation of Nuclear Factor Erythroid 2-Related Factor 2 (Nrf2), thereby expressing antioxidant proteins that protect against ROS.

In the compositions and methods herein, the ophthalmic formulation comprises EGT and the pharmaceutically acceptable aqueous delivery vehicle, wherein the pharmaceutically acceptable aqueous delivery vehicle is prepared by step-by-step dissolutions of excipients selected from the group consisting of a tonic agent, buffer, preservative, viscosity enhancer, stabilizer, pH adjuster, penetration enhancer, local anesthetic, restorative agent, surfactant, and thickeners. Sterilization and strict processing of the pharmaceutically acceptable aqueous delivery vehicle ensure the functionality and sterility of the excipients in the pharmaceutically acceptable aqueous delivery vehicle, to which EGT or derivatives of EGT is added. This process balances the stability of active ingredients, preparation safety and production efficiency, and complies with the requirements of Good Manufacturing Practice (GMP) (current Good Manufacturing Practice cGMP) for pharmaceutical production.

In the compositions and methods herein, the preferred embodiment of the ophthalmic formulations herein contains L-Ergothioneine (EGT), as represented by Formula 1. L-Ergothioneine is a derivatized histidine, wherein the imidazole is disubstituted at the 2-position and the 4-position and the amine is trimethylated. More specifically, the imidazole (C(NH)(NH)(CHC)) is sulfurized (S) at the 2-position and connected to methylene (CH₂), wherein methylene is connected to a methine group (CH), wherein methine is connected to trimethylammonium (N(CH₃)₃) and carboxylate (CO₂). The trimethylammonium group, which is cationic by virtue of being a quaternary amine (without any protons attached to the nitrogen); and the carboxylate, which is anionic by virtue of being in a deprotonated state, lead a betaine construct for promoting surfactant and emulsification properties. Surfactant and emulsification properties aid in: (1) dissolving L-Ergothioneine in water of the ophthalmic formulation; and (2) releasing L-Ergothioneine from the ophthalmic formulation, when introduced into ocular tissue. At the 2-position of imidazole, sulfur can be in thione form (C═S) or thiolate (C—S—) or thiol (C—SH) forms. However, thione is the favored canonical structure in the ophthalmic formulation and in the ocular tissue, when the formulation containing L-Ergothioneine is introduced into the ocular tissue. In thione form, L-Ergothioneine is able to scavenge reactive species and bind to metal centers (e.g., free ion or accessible faces of a transition metal complex). The antioxidant properties of thione-imidazole (i.e., chemo-selective oxidation of the sulfur of the thione-imidazole) and binding behavior of L-Ergothioneine in thione form to metal centers can allow for a controlled mechanism to be operable in the ocular tissue, when introduced therein from the ophthalmic formulation.

S(C(NH)(NH)(CHC))—CH₂—CH(N(CH₃)₃)(CO₂) [Formula 1]

In the compositions and methods herein, other embodiments of the ophthalmic formulation includes derivatives of L-Ergothioneine, as represented by Formula 2. Derivatives of L-Ergothioneine comprise imidazole that is disubstituted at the 2-position and the 4-position and the amine is trimethylated. More specifically, the imidazole (C(N)(NH)(CHC)) is connected to SR at the 2-position and connected to methylene (CH₂), wherein methylene is connected to a methine group (CH), wherein methine is connected to trimethylammonium (N(CH₃)₃) and carboxylate (CO₂). The trimethylammonium group, which is cationic by virtue of being a quaternary amine (without any protons attached to the nitrogen); and the carboxylate, which is anionic by virtue of being in a deprotonated state, lead a betaine construct for promoting surfactant and emulsification properties. Surfactant and emulsification properties aid in: (1) dissolving the derivatives of L-Ergothioneine in water of the ophthalmic formulation; and (2) releasing the derivatives of L-Ergothioneine from the ophthalmic formulation, when introduced into ocular tissue.

RS(C(N)(NH)(CHC))—CH₂—CH(N(CH₃)₃)(CO₂) [Formula 2]

In the compositions and methods herein, at the 2-position of imidazole, the R group of SR of the derivatives of L-Ergothioneine are functional groups that make thioethers, where: R═CH₃, CH₂CH₃, CH₂CH₂CH₃, CH₃CHCH₃, CH₂—X, and X=1-indole, 4-Indole, 7-indole, 1-imidazole, 2-imidazole, 3-imidazole, 4-imidazole, 5-imidazole, benzene, 2-oxazole, 3-oxazole, 4-oxazole, 2-thiazole, 3-thiazole, 4-thiazole, 2-pyridine, 6-pyridine, 1-pyrrole, 2-pyrrole, and 3-pyrrole. While the connectivity of sulfur to the imidazole in Formula 2 does not make a thione connectivity as a feasible canonical structure, the thioethers of Formula 2 do not yield a thiolate or thiyl. In thioether form, L-Ergothioneine is able to scavenge reactive species and bind to metal centers (e.g., free ion or accessible faces of a transition metal complex). The antioxidant properties of thioether-imidazole (i.e., chemo-selective oxidation of the sulfur of the thioether-imidazole) and binding behavior of the derivatives of L-Ergothioneine in thioether form to metal centers can allow for a controlled mechanism to be operable in the ocular tissue, when introduced therein from the ophthalmic formulation.

In the compositions and methods herein, at the 2-position of imidazole, the R group of SR of the derivatives of L-Ergothioneine is not H. Thus, sulfhydryl (SH) is not attached to the 2-position of imidazole. Sulfhydryl can donate a proton (H⁺) to yield a thiolate anion (—S—(C(N)(NH)(CHC))—) or hydrogen radical (H•) to yield thiyl radical (•S—(C(N)(NH)(CHC))—). Thiolate and thiyl are canonical structures that are far more reactive than the thione, and therefore not as easy to control the reactivity, in comparison to the thione form. This can lead to redox processes that can reduce or entirely suppress the operability of the controlled mechanism, as indicated above.

### EXAMPLES

The following illustrative examples are representative of embodiments of the compositions, systems, and methods described herein that have been reduced practice, and are not meant to be limiting in any way.

### Example 1—Formulations, where EGT is an API

The ophthalmic formulation comprised: 0.5% (w/v) EGT as an active pharmaceutical ingredient (API) with: 0.05% (w/v), 0.01% (w/v), or 0% (w/v) of sodium hyaluronate; almost negligible amounts of taurine (0.01% (w/v), 0.03% (w/v), or 0.05% (w/v) of taurine); 0.5-0.9% (w/v) of sodium chloride; and the remainder is the pharmaceutically acceptable aqueous delivery vehicle, such that the overall water content of the formulation is 98-99.5% (w/v). In all the cases, sodium ions, from sodium hyaluronate and sodium chloride, were used to adjust the osmotic pressure of eye drops to the regular level. In the formulations that comprised 0.5% (w/v) EGT exhibited: high ROS scavenging efficiency; high potency; and no detectable irritation in the ocular tissue of the subjects. There were 88 subjects with dry eye, which manifested as one or more of the following: eye dryness, eye itching, eye redness, foreign body sensation, photophobia/wind sensitivity with tearing, sticky secretions, blurred vision, and painful sensation. The subjects were prompted to indicate the level of improvement of their symptoms related to dry eyes after consuming the formulation comprising EGT and whether the formulation comprising EGT provided a better experience than other eye drops or eye wash products. Of the 88 subjects, 20 subjects reported very obvious improvement in symptoms after using the formulation; 50 subjects reported obvious improvement in symptoms after using the formulation; 12 subjects reported some improvements in symptoms after using the formulation; 4 subjects reported not obvious improvements in symptoms after using the formulation; and 2 subjects reported there were little or no improvements in symptoms after using the formulation. Of the 88 subjects, 24 reported that the formulation was much better than prior used eye products; 51 reported that the formulation was better than prior used eye products; 13 reported that the formulation was the same as prior used eye products; and 0 reported that the formulation was not as good as prior used eye products.

### Example 2—Trial of L-EGT Eye Wash for Improving Ocular Discomfort

Two eye washes (or ophthalmic formulations) were prepared and investigated to: (1) study the effectiveness of EGT for improving ocular discomfort compared with the self-control group; and (2) observe the safety of EGT during the clinical trial. Eye Wash 1 was 5.0 mL (water, 0.5% EGT, sodium hyaluronate, taurine, and sodium chloride); and wash 2 was 0.5 mL (water, 0.5% EGT, sodium hyaluronate, taurine, and sodium chloride). Forty subjects completed the trial, where 20 subjects used wash 1 and 20 subjects used wash 2. In the safety data set of this trial, a total of 0 subjects experienced 0 adverse events, with an incidence rate of adverse events of 0%. No subject had adverse reactions or serious adverse events. The research methodology was: a single-center, randomized, double-blind, parallel, placebo-controlled clinical.

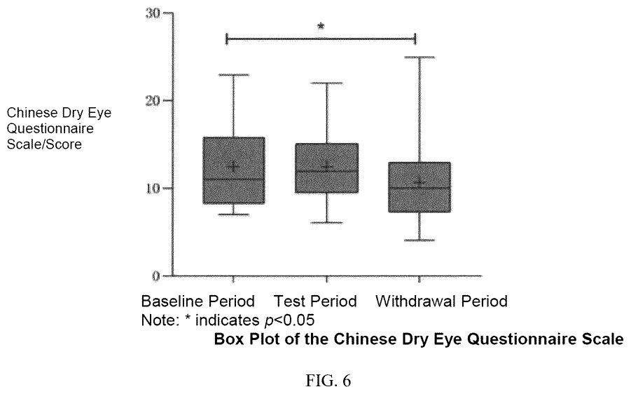

The mean value of the questionnaire was 12.50, and the mean value at the end-of-study period was 10.65, which decreased by 14.80%, and there was significant difference (P=0.0353), as depicted in FIG. 6. After the subjects were administered with EGT Eye Wash, the mean score of the Questionnaire Scale in the post-treatment period was significantly lower than that in the baseline period (P=0.0353).

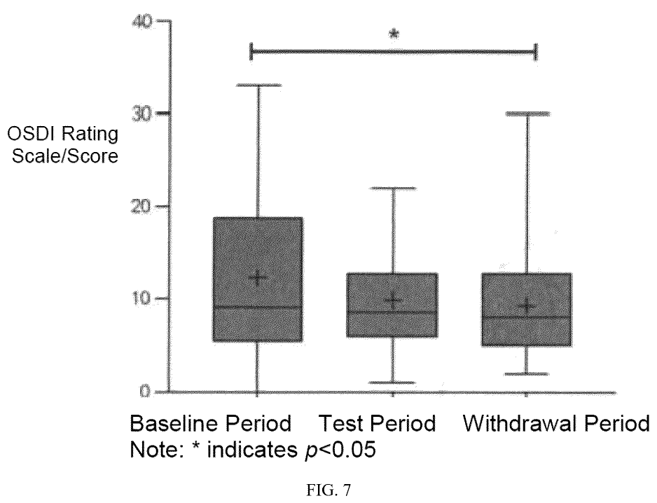

The mean value of the Ocular Surface Disease Index (OSDI) score of 40 subjects at the baseline period was 12.25, the mean value at the withdrawal period was 9.23, which decreased by 24.69%, and there was a significant difference (P=0.0309), as depicted in FIG. 7. After the subjects were administered with EGT Eye Wash, the mean score of the Ocular Surface Disease Index (OSDI) in the post-treatment period was significantly lower than that in the baseline period (P=0.0309).

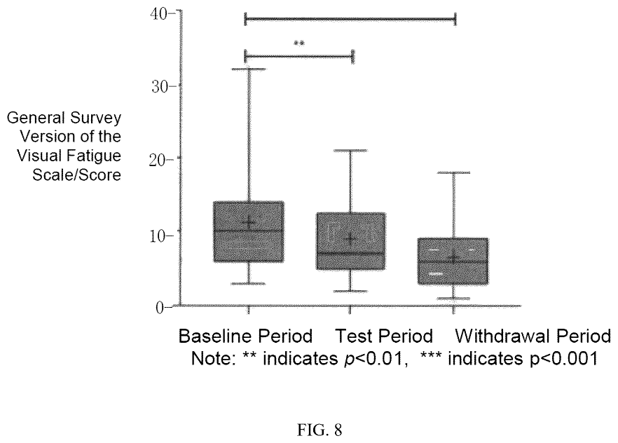

The mean baseline value of the scores of the General Survey Version of the Visual Fatigue Scale for 40 subjects was 11.18, and the mean value at the withdrawal period was 6.60, representing a decrease of 40.94%. There was a significant difference (P=0.0008), as depicted in FIG. 8. After the subjects were administered with the EGT Eye Wash, the mean score of the General Survey Version of Visual Fatigue Scale in the post-treatment period was significantly lower than that in the baseline period (P=0.0008).

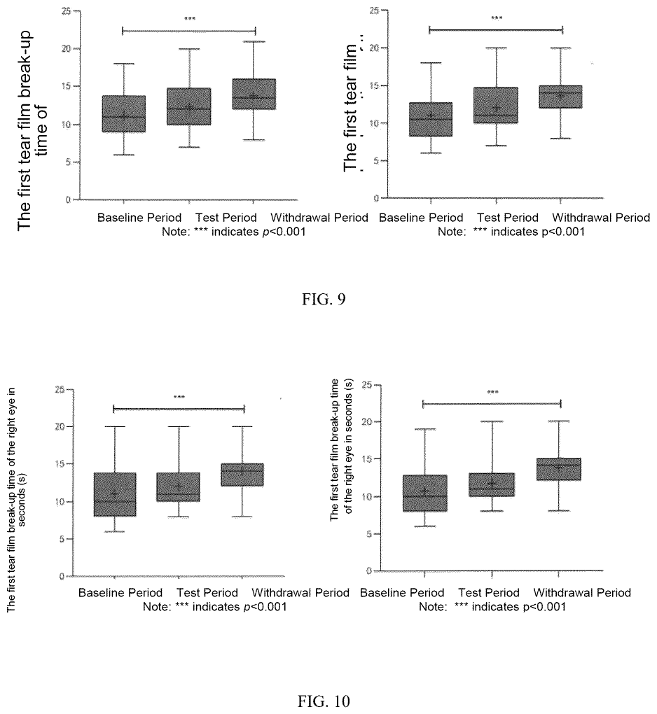

For the 40 subjects, at the withdrawal period, the mean values of the first tear film break-up time of the left eye, the average tear film break-up time of the left eye, the first tear film break-up time of the right eye, and the average tear film break-up time of the right eye increased by 23.60%, 24.56%, 26.98%, and 27.74% respectively compared with those at the baseline period. All of these showed statistically significant differences (P<0.0001, P<0.0001, P<0.0001, P<0.0001), as depicted in FIG. 9 (left eye) and FIG. 10 (right eye). After the subjects were administered with the EGT Eye Wash, the mean values of the first tear film break-up time and the average tear film break-up time of both eyes in the post-treatment period was significantly higher than those in the baseline period (P<0.05).

### Example 3—Formulations

The pharmaceutically acceptable aqueous delivery vehicle was prepared by mixing excipients in water and sterilizing the resulting aqueous solution. EGT was added to the aqueous solution to make eye drops that were 98.0 to 99.5% (w/v) of water. Eye drops of the formulation herein have 0.30%-0.75% (w/v) of EGT, 0.5%-1.0% (w/v) of EGT; 1.0%-2.0% (w/v) of EGT; 0.005% to 0.5% (w/v) of EGT; or 0.005%-0.30% (w/v) of EGT

### Formulation 1

| Component | Amount (% (w/v)) |
|---|---|
| EGT | 0.50-1.00 |
| Aqueous Delivery Vehicle | 98.50-99.0 |

### Formulation 2

| Component | Amount (% (w/v)) |
|---|---|
| EGT | 0.30-0.75 |
| Water | 98.50-99.00 |
| Excipients | 0.25-1.20 |

### Formulation 3

| Component | Amount (% (w/v)) |
|---|---|
| EGT | 0.50 |
| Glutathione | 0.10-0.50 |
| Water | 98.50-99.00 |
| Excipients | 0.40-0.50 |

### Formulation 4

| Component | Amount (% (w/v)) |
|---|---|
| EGT | 0.50 |
| Lutein | 0.10-0.50 |
| Water | 98.50-99.00 |
| Excipients | 0.40-0.50 |

### Formulation 5

| Component | Amount (% (w/v)) |
|---|---|
| EGT | 0.50 |
| EDTA | 0.01-0.10 |
| Water | 98.50-99.00 |
| Excipients | 0.40-0.50 |

### Formulation 6

| Component | Amount (% (w/v)) |
|---|---|
| EGT | 0.50 |
| Sodium hyaluronate | 0.05 |
| Taurine | 0.010 |
| Sodium chloride | 0.50-0.90 |
| Water or Aqueous Delivery Vehicle | 98.54-98.84 |

### Formulation 7

| Component | Amount (% (w/v)) |
|---|---|
| EGT | 0.50 |
| Sodium hyaluronate | 0.050 |
| Taurine | 0.03 |
| Sodium chloride | 0.50-0.90 |
| Water or Aqueous Delivery Vehicle | 98.52-98.82 |

### Formulation 8

| Component | Amount (% (w/v)) |
|---|---|
| EGT | 0.50 |
| Sodium hyaluronate | 0.05 |
| Taurine | 0.05 |
| Sodium chloride | 0.50-0.90 |
| Water or Aqueous Delivery Vehicle | 98.50-98.80 |

1. An ophthalmic formulation comprising:
a pharmaceutically acceptable aqueous delivery vehicle comprises:
an active ingredient with biological activity or therapeutic effect associated with a labile drug introduced into ocular tissue, wherein the active ingredient is spermine and wherein spermine is present in an amount of 0.01-0.1% (w/v) in the ophthalmic formulation;
L-Ergothioneine (EGT) in a concentration of 0.005% to 0.5% (w/v) of the formulation, wherein the EGT is present in a concentration wherein the EGT behaves as an excipient that:
inhibits degradation of the active ingredient introduced into the ocular tissue, and
inductively expresses OCTN1, thereby increasing bioavailability of the active ingredient.

2. (canceled)

3. The formulation of claim 1, wherein EGT scavenges hydroxyl and peroxynitrite via a non-enzymatic redox cycle, such that: the hydroxyl is reduced to water and EGT is oxidized to a sulfate; and the peroxynitrite is reduced to nitrate and EGT is oxidized to a sulfate.

4. The formulation of claim 1, wherein EGT coordinates to a metal selected from iron, copper, and other transition metals in an ocular surface of the ocular tissue, thereby inhibiting catalysis of oxidative degradation of the labile drug by:
forming a complex between EGT and the metal, wherein the metal is in a reduced state; and
reducing an oxidation potential of the metal due to the formation of the complex, thereby suppressing formation of metal-oxygen adducts for deriving reactive oxygen species;
wherein the complex comprises: (i) two units of EGT binding to 1 unit of the metal or (ii) 1 unit of EGT and 1 unit of an initial ligand core binding to 1 unit of the metal.

5. The formulation of claim 1, wherein EGT in the formulation is reducing reactive oxygen species (ROS) in corneal epithelial cells.

6. (canceled)

7. The formulation of claim 1, wherein the ocular tissue has conditions associated with glaucoma.

8. The formulation of claim 1, wherein OCTN1 transports the active ingredient within the ocular tissue.

9. The formulation of claim 1, further comprises one or more of: glutathione, ubiquinone, lutein; and ethylenediaminetetraacetic acid.

10. The formulation of claim 1, wherein EGT has a chemical formula of C₉H₁₅N₃O₂S, where the carbon-sulfur bond is in thione form (C═S) over thiolate form (C—S⁻), in the pharmaceutically acceptable aqueous delivery vehicle.

11. The formulation of claim 1, wherein said formulation further comprises:
0.05% (w/v); 0.01% (w/v), or 0% (w/v) of sodium hyaluronate;
trace amounts of taurine;
sodium chloride for adjusting the osmotic pressure of eye drops; and
the pharmaceutically acceptable aqueous delivery vehicle.

12. An ophthalmic formulation comprising:
a pharmaceutically acceptable aqueous delivery vehicle; and
a derivative of L-Ergothioneine (EGT) in a concentration of 0.005% to 3.000% (w/v) in the pharmaceutically acceptable aqueous delivery vehicle, wherein the derivative of EGT is:
an active ingredient for treating an oxidative stress-related ocular disorder or disease; or
an inactive excipient to inhibit degradation of a co-formulated active ingredient;
wherein the derivative of EGT is: 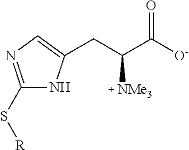
where R=CH₃, CH₂CH₃, CH₂CH₂CH₃, CH₃CHCH₃, CH₂—X, and X=1-indole, 4-Indole, 7-indole, 1-imidazole, 2-imidazole, 3-imidazole, 4-imidazole, 5-imidazole, benzene, 2-oxazole, 3-oxazole, 4-oxazole, 2-thiazole, 3-thiazole, 4-thiazole, 2-pyridine, 6-pyridine, 1-pyrrole, 2-pyrrole, and 3-pyrrole.

13. The formulation of claim 12, wherein the co-formulated active ingredient is selected from the group consisting of: nucleotides, acetylcholine, tetraethylammonium chloride, spermine, L-carnitine, sulpiride, homostachydrine, cytarabine, gemcitabine, gabapentin, oxaliplatin, metformin, cyclosporine, and lifitegrast.

14. The formulation of claim 12, wherein the derivative of EGT scavenges hydroxyl, such that the hydroxyl is reduced to water and the derivative of EGT is oxidized to a sulfoxide.

15. The formulation of claim 12, wherein the derivative of EGT as the inactive excipient in the formulation is decreasing reactive oxygen species (ROS) in corneal epithelial cells.

16. The formulation of claim 12, wherein:
the derivative of EGT is 1.0% to 3.0% (w/v) of the formulation, when functioning as an active ingredient; and
the derivative of EGT is 0.005% to 1.0% (w/v), when functioning as an inactive excipient.

17. The formulation of claim 12, wherein the oxidative stress-related ocular disorder or disease comprises: allergies, irritated eyes, red eye, age-related macular degeneration; cataracts; glaucoma; diabetic retinopathy; dry eye syndrome; and retinitis pigmentosa.

18. A method of treating an oxidative stress-related ocular condition, disorder, or disease in a subject, wherein the method comprises:
administering a formulation to said subject, wherein the formulation comprises the ophthalmic formulation of claim 1.

19. The method of claim 18, wherein EGT or the derivative of EGT scavenges reactive oxygen species via a non-enzymatic redox cycle.

20. The method of claim 18, wherein EGT or the derivative of EGT coordinates to a metal selected from iron, copper, and other transition metals in an ocular surface, thereby inhibiting catalysis of oxidative degradation of labile drugs and decreasing reactive oxygen species (ROS) in corneal epithelial cells.
